(19) United States
(12) Patent Application Publication
(10) Pub. No.: US 2025/0057907 A1
(43) Pub. Date: Feb. 20, 2025

(54) Antidepressant, Anti-Anxiety, Anti-Stress, or Tension-Relieving Composition Containing Fermented Perilla Frutescens Extract, and Use Thereof

(71) Applicant: Ajou University Industry-Academic Cooperation Foundation, Suwon-si, Gyeonggi-do (KR)

(72) Inventors: Yi Sook Jung, Yongin-si, Gyeonggi-do (KR); Tae Ho Kim, Pyeongtaek-si, Gyeonggi-do (KR)

(21) Appl. No.: 18/716,747

(22) PCT Filed: Feb. 19, 2020
(86) PCT No.: PCT/KR2020/002397

§ 371 Date: Nov. 7, 2024

(30) Foreign Application Priority Data
Mar. 15, 2019 (KR) 10-2019-0029789

(51) Int. Cl. A61K 36/535; A23L 2/52; A61K 8/9789; A61P 25/22; A61Q 19/00; C11B 9/02

(52) U.S. Cl. CPC A61K 36/535; A23L 2/52; A61K 8/9789; A61P 25/22; A61Q 19/00; C11B 9/02; A61K 2236/19; A61K 2236/331; A61K 2800/85

(57) ABSTRACT

The present invention relates to an antidepressant, anti-anxiety, anti-stress, tension-relieving or stress-relieving composition containing a fermented Perilla frutescens extract. In addition, the present invention relates to a fermented Perilla frutescens extract-containing composition for preventing, ameliorating, or treating stress-related disorders, depression, or anxiety. In addition, the present invention relates to a fermented Perilla frutescens extract-containing fiber or fragrance composition for relieving depression, anxiety, tension, or stress. In addition, the present invention relates to a method for treating depression or anxiety, the method comprising administering a fermented Perilla frutescens extract to a patient suffering from a stress-related disorder, depression, or anxiety. A fermented Perilla frutescens (Perilla frutescens Britton var. acuta Kudo (Chajgi), or Perilla frutescens Britton var. crispa Decaisn (Jasoyeop)) extract according to the present invention significantly relieves stress compared to an unfermented Perilla frutescens extract, and thus can be used as an antidepressant, anti-anxiety, tension-relieving or stress-relieving composition, and furthermore, can be effectively used in a composition or therapeutic method for preventing, ameliorating, or treating stress-related disorders, depression, and anxiety.

### TECHNICAL FIELD

This application claims the priority of Korean Patent Application No. 10-2019-0029789, filed on Mar. 15, 2019, the entirety of which is a reference of the present application.

The present invention or application is further supported by the Ministry of Science and ICT and the Agency for the Promotion of Science and Technology Commercialization (IP Star Scientist Support Type) of University Technology Management. [Task unique number] RS-2024-00425768. [Ministry Name] Ministry of Science and ICT. [Research Management Agency] Agency for the Promotion of Science and Technology Commercialization. [Research project name] Promotion of university technology (IP Star Scientist Support Type). [Research assignment name] IP advancement and commercialization of natural factor technology to improve stress/sleep quality. [Host organization] AJOU UNIVERSITY INDUSTRY-ACADEMIC COOPERATION FOUNDATION. [Research Period] 2024 Apr. 1˜2025 Dec. 31.

The present invention relates to an antidepressant, anti-anxiety, anti-stress, tension-relieving or stress-relieving composition containing a fermented Perilla frutescens extract.

In addition, the present invention relates to a fermented Perilla frutescens extract-containing composition for preventing, ameliorating or treating stress-related disorders, depression or anxiety.

In addition, the present invention relates to a fermented Perilla frutescens extract-containing fiber or fragrance composition for relieving depression, anxiety, tension, or stress.

In addition, the present invention relates to a method for treating stress-related disorders, depression or anxiety, including administering a fermented Perilla frutescens extract to a patient suffering from stress-related disorders, depression, or anxiety.

### BACKGROUND ART

As society develops and diversifies rapidly, modern people are surrounded by various types of stress. Failure to successfully cope with stress results in psychological reactions such as depression, anxiety, fatigue, anger, and mood changes, as well as physiological reactions such as decreased «-waves in brain waves and increased blood pressure and pulse rate. However, when these reactions continue to be repeated, diseases such as depression and anxiety occur to cause personal, family, and social losses, and lower the individual's quality of life. Recently, mental disorders in adolescents are also on the rise due to causes such as excessive desire to learn.

According to “Survey of Mental Disorders in Korea 2016” published by the Ministry of Health and Welfare, the ‘annual prevalence rate of mental disorders’, which is the proportion of the population experiencing one or more mental disorders in one year, is 12.1%, which is 1 of about 8 adults between the ages of 18 and 64 and ‘the lifetime prevalence rate of mental disorders’, which is the proportion of the population experiencing one or more mental disorders during their lifetime, is 26.6%, which is 1 of about 4 adults. The incidence rate is increasing worldwide, and the World Health Organization predicted that depression would become the most common disease among diseases at all ages in coming 2020. Nevertheless, only 18.2% of people diagnosed with mental disorders have any experience with counseling or treatment for mental problems.

Among mental disorders caused by stress, depression refers to a psychological state of depressed mood characterized by feelings of sadness, hopelessness, and frustration. The depression refers to a condition that progresses from the normal emotion of “gloomy” to major depressive disorder via dysthymic disorder. Symptoms of the depression include major depressive episodes, which include changes in appetite and weight patterns, psychomotor agitation or retardation, decreased thinking ability, concentration or decision ability, lack of vitality, fatigue, worthlessness, remorse, or guilty conscience, frequent thoughts of death or suicide, plans or attempts to commit suicide, low mood in daily life, loss of interest or pleasure in most activities, etc.

There are various hypotheses about the biological cause of depression, but there is a prevailing view that the biological cause is determined as disorders of the brain neurotransmitter system such as adrenaline, dopamine, or serotonin. Currently, tricyclic antidepressants (TCAs) or selective serotonin re-uptake inhibitors (SSRIs) are mainly used as therapeutic agents for depression, but the SSRIs have the problem of being less effective and taking a long time. According to the American Psychiatric Association (APA) recommendations, the SSRIs should be taken for at least 4 months, and may extend to 2 to 3 years depending on the patient. In addition, it is known that there is a risk that most depression symptoms will recur within 6 to 12 months after stopping the drug.

Most drugs used for mental disorders are classified as psychotropic drugs, and particularly, benzodiazepine drugs act directly on gamma-aminobutyric acid (GABA) receptors in the central nervous system, causing excessive sedation, and have a high risk of drug abuse due to high psychological or physical dependence.

Meanwhile, Perilla frutescens as an annual plant in the family Lamiaceae is used to treat various diseases such as cough, phlegm, tonsillitis, indigestion, boils, paralysis, diabetes, and lumbago, and known to have antibacterial and anticancer effects.

Japanese Patent Application No. 2004-219382 relates to a composition exhibiting actions of promoting good sleep and a drink containing the same, and discloses that the composition includes a Perilla frutescens leave extract, and when consumed, the composition has an effect of inducing good sleep, such as extending sleeping time. However, no research or report has recently been conducted on the effects of a Perilla frutescens extract fermented with a microorganism or a composition thereof on relieving depression, anxiety, tension or stress.

### DISCLOSURE

### Technical Problem

Therefore, the present inventors have made intensive efforts to provide an effective natural material without side effects on mental disorders related to depression, anxiety, tension or stress, etc., and as a result, confirmed that a fermented Perilla frutescens extract had an excellent stress-relieving effect and may replace existing drugs that have been used to treat mental disorders, and then completed the present invention.

Accordingly, an object of the present invention is to provide an antidepressant, anti-anxiety, anti-stress, tension-relieving or stress-relieving composition containing a fermented Perilla frutescens extract.

Another object of the present invention is to provide a fermented Perilla frutescens extract-containing composition for preventing, ameliorating, or treating stress-related disorders, depression, or anxiety.

Yet another object of the present invention is to provide a fermented Perilla frutescens extract-containing fiber or fragrance composition for relieving depression, anxiety, tension, or stress.

Still another object of the present invention is to provide a method for treating stress-related disorders, depression or anxiety, including administering a fermented Perilla frutescens extract to a patient suffering from stress-related disorders, depression, or anxiety.

### Technical Solution

One aspect of the present invention provides an antidepressant, anti-anxiety, anti-stress, tension-relieving or stress-relieving composition containing a fermented Perilla frutescens extract.

According to a preferred embodiment of the present invention, the fermented extract may be extracted from at least one part selected from the group consisting of leaves, stems, flowers, fruits, and whole plant of Perilla frutescens.

According to a preferred embodiment of the present invention, the fermented extract may be extracted using water, an organic solvent, or a mixture thereof as a solvent and then fermented with a fermentation strain.

According to a preferred embodiment of the present invention, the fermentation strain may be Bacillus or Lactobacillus bacteria.

According to a preferred embodiment of the present invention, the Bacillus bacteria may be at least one strain of the genus Bacillus selected from the group consisting of Bacillus subtilis, Bacillus thuringiensis, Bacillus licheniformis, Bacillus amyloliquefaciens, Bacillus stearothermophilus, Bacillus coagulans, Bacillus longum, Bacillus pumilus, Bacillus brevis, Bacillus circulans, and Bacillus polymyxa; and the Lactobacillus bacteria may be at least one of the genus Lactobacillus selected from the group consisting of Lactobacillus acidophilus, Lactobacillus casei, Lactobacillus gasseri, Lactobacillus bulgaricus, Lactobacillus helveticus, Lactobacillus fermentum, Lactobacillus paracasei, Lactobacillus plantarum, Lactobacillus reuteri, Lactobacillus rhamnosus, and Lactobacillus salivarius.

According to a preferred embodiment of the present invention, the fermentation may be performed at 5° C. to 80° C. for 30 minutes to 10 days.

According to a preferred embodiment of the present invention, the depression, anxiety or tension may be caused by stress.

According to a preferred embodiment of the present invention, the composition may be at least one composition selected from the group consisting of a food composition, a cosmetic composition, a dye composition, a pharmaceutical composition, and a quasi-drug composition.

Another aspect of the present invention provides a fermented Perilla frutescens extract-containing composition for preventing, ameliorating or treating stress-related disorders, depression or anxiety.

According to a preferred embodiment of the present invention, the stress-related disorders may be at least one selected from the group consisting of stress tension, stress anxiety, stress depression, fatigue syndrome, fear memory, mental panic attack, obsessive-compulsive disorder, post-traumatic stress disorder, schizophrenia, stress headaches, neurodegenerative diseases, eating disorders, anorexia nervosa, drug addiction, drug or alcohol withdrawal syndromes, and stress psychotic episodes.

Yet another aspect of the present invention provides a fermented Perilla frutescens extract-containing fiber or fragrance composition for relieving depression, anxiety, tension, or stress.

According to a preferred embodiment of the present invention, the fiber may be dyed with the fermented Perilla frutescens extract.

According to a preferred embodiment of the present invention, the fiber may be included in at least one product selected from the group consisting of bedding, clothing, curtains, carpets, indoor shoes, towels, wallpaper, indoor cover products made of fiber materials, dolls, and eye masks.

According to a preferred embodiment of the present invention, the fragrance composition may be included in at least one product selected from the group consisting of perfumes, incense, oils, air fresheners, detergents, and skin external agents.

Still another aspect of the present invention provides a method for treating stress-related disorders, including administering a fermented Perilla frutescens extract to a patient suffering from stress-related disorders.

Still another aspect of the present invention provides a method for treating depression or anxiety, including administering a fermented Perilla frutescens extract to a patient suffering from depression or anxiety.

As described above, drugs used in the prior art to relieve or treat stress or mental disorders, etc. had limitations of causing excessive sedation, dependence, and side effects. In order to replace the drugs, there is a need to develop a composition that has excellent effects in alleviating or treating stress, mental disorders, etc. even while using plant-derived natural products.

The fermented Perilla frutescens extract according to the present invention is obtained by fermenting an extract derived from Perilla frutescens using various microorganisms such as Bacillus subtilis, and thus has the effect of significantly reducing anxiety, depression, tension, or stress. Thus, as the antidepressant, anti-anxiety, anti-stress, tension-relieving or stress-relieving composition, the fermented Perilla frutescens extract may also be used as a composition for preventing, ameliorating, or treating stress-related disorders, depression, and anxiety. Furthermore, the fermented Perilla frutescens extract may also be used as a method for treating stress-related disorders, depression or anxiety by including administering the fermented Perilla frutescens extract to a patient suffering from stress-related disorders, depression, or anxiety.

The “fermented Perilla frutescens extract” of the present invention means an extract obtained by first preparing a Perilla frutescens extract and then fermenting the extract using a fermentation strain.

The “stress-related disorders” of the present invention refer to diseases that may be caused by mental or physical stress, and a concept including all of stress tension, stress anxiety, stress depression, fatigue syndrome, fear memory, mental panic attack, obsessive-compulsive disorder, post-traumatic stress disorder, schizophrenia, stress headaches, neurodegenerative diseases, eating disorders, anorexia nervosa, drug addiction, drug or alcohol withdrawal syndromes, and stress psychotic episodes.

The “depression (depressive disorder)” of the present invention refers to a disease that causes various cognitive, mental, or physical symptoms with decreased motivation and depression as main symptoms, resulting in a decline in daily function. The causes are very diverse chemically, biologically, psychologically, sociologically, pharmacologically, or pathologically, and one of the causes is a chemical imbalance of neurotransmitters such as serotonin, melatonin, dopamine, or norepinephrine. In addition, the depression may be caused by chronic stress, taking drugs such as antihypertensives, anti-anxiety drugs, narcotics, and central nervous system stimulants, or diseases such as diabetes, pancreatic cancer, and endocrine diseases.

The “anxiety (anxiety disorder)” of the present invention refers to a mental disorder that exhibits extreme anxiety such as nervousness, tension, and worry that is continuous and unrecoverable for usually more than 6 months. The causes are very diverse, but medically, have been reported to be caused by a lack or excess of neurotransmitters in the brain nerves, genetic predisposition, and functional or structural abnormalities in the brain revealed through brain imaging studies, and constant stress may also be an important cause. Particularly, in children and adolescents, when attention deficit hyperactivity disorder (ADHD) is severe, the ADHD may lead to anxiety and depression. The anxiety is broadly classified into four types: generalized anxiety disorder (GAD), obsessive-compulsive disorder, panic disorder, and post-traumatic stress disorder, and other types include acute stress disorder, separation anxiety disorder, etc.

The “relieving” of the present invention means all effects of qualitatively or quantitatively ameliorating symptoms related to depression, anxiety or tension and other stress or stress-induced symptoms.

When the Perilla frutescens extract was administered to mice with a depression, anxiety, tension or stress condition without fermentation, the condition was not relieved, but when the Perilla frutescens extract was administered to mice with the depression, anxiety, tension or stress condition after fermentation, it was confirmed that the condition was significantly relieved (FIGS. 1 to 4).

Particularly, after the fermented Perilla frutescens extract prepared by fermenting the Perilla frutescens extract with Bacillus subtilis or Lactobacillus rhamnosus (Example 1) is administered to mice that were induced with depression, anxiety, tension or stress by restraint or sleep deprivation, as a result of performing a tail suspension test or a forced swimming test (Examples 2 and 3), the immobility time of the mice was significantly reduced. This means that the fermented Perilla frutescens extract of the present invention has an effect of relieving depression, anxiety, tension or stress.

Accordingly, an aspect of the present invention may provide an antidepressant, anti-anxiety, anti-stress, tension-relieving or stress-relieving composition containing a fermented Perilla frutescens extract.

The Perilla frutescens is also called Perilla frutescens Britton var. acuta Kudo (Chajgi) or Perilla frutescens Britton var. crispa Decaisn (Jasoyeop), and is known to have antibacterial and anticancer effects. The Perilla frutescens is also used in rice dishes such as California rolls or Sushi, and is also used as a dye to make the color of food or dye fabrics.

To prepare the fermented Perilla frutescens extract, first, the Perilla frutescens extract is prepared. The Perilla frutescens extract may be extracted from the aerial parts such as leaves, stems, flowers or fruits or whole plant of Perilla frutescens, but is preferably extracted from the aerial parts such as leaves, stems, flowers or fruits of Perilla frutescens. The extraction solvent may be water, an organic solvent or a mixture thereof, but preferably water or C₁-C₄ lower alcohols, more preferably water, methanol, ethanol or propanol, and most preferably water as the solvent (Example 1).

The fermentation strain that may be used to ferment the Perilla frutescens extract may be Bacillus or Lactobacillus bacteria. Preferably, the Bacillus bacteria are the genus Bacillus and the Lactobacillus bacteria are the genus Lactobacillus to enhance an antidepressant, anti-anxiety or tension-relieving effect of the Perilla frutescens extract.

The Bacillus bacteria may be at least one strain of the genus Bacillus selected from the group consisting of Bacillus subtilis, Bacillus thuringiensis, Bacillus licheniformis, Bacillus amyloliquefaciens, Bacillus stearothermophilus, Bacillus coagulans, Bacillus longum, Bacillus pumilus, Bacillus brevis, Bacillus circulans, and Bacillus polymyxa; and the Lactobacillus bacteria may be at least one strain of the genus Lactobacillus selected from the group consisting of Lactobacillus acidophilus, Lactobacillus casei, Lactobacillus gasseri, Lactobacillus bulgaricus, Lactobacillus helveticus, Lactobacillus fermentum, Lactobacillus paracasei, Lactobacillus plantarum, Lactobacillus reuteri, Lactobacillus rhamnosus, and Lactobacillus salivarius.

The fermentation conditions are preferably performed at 5° C. to 80° C. for 30 minutes to 10 days, more preferably at 20° C. to 40° C. for 3 to 8 days, and most preferably at 22° C. to 30° C. for 4 to 6 days (Example 1).

The administration route of the fermented Perilla frutescens extract can be oral or parenteral, and in the case of parenteral administration, the fermented Perilla frutescens extract may be administered through external skin application, injection, transdermal or nasal route, or the like, but is not limited thereto. The dose may be administered at 0.001 mg to 10,000 mg per 1 kg, preferably at 0.01 mg to 1,000 mg per 1 kg, but is not limited thereto.

The composition of the present invention has an effect of relieving depression, anxiety or tension caused by various causes, but preferably may have an effect of relieving depression, anxiety or tension caused by stress.

The composition of the present invention may be at least one composition selected from the group consisting of a food composition, a cosmetic composition, a dye composition, a pharmaceutical composition, and a quasi-drug composition, but is not limited thereto.

When formulated as the cosmetic composition, the content of the fermented Perilla frutescens extract is 0.0001 to 10 wt %, preferably 0.01 to 5.0 wt %, based on the total weight of the cosmetic composition. It is preferable that the content of the fermented Perilla frutescens extract is the minimum value or more to achieve at least an effect of ameliorating or preventive skin cancer, and it is preferable that the content of the fermented Perilla frutescens extract is the maximum value or less by considering decreased usability due to excessive addition and applicability to various formulations. At this time, it is preferable to appropriately adjust the content of the fermented Perilla frutescens extract within the range depending on the content of ingredients contained in the formulation or cosmetic composition.

The ingredients included in the cosmetic composition of the present invention include ingredients commonly used in the cosmetic composition in addition to the fermented Perilla frutescens extract as an effective ingredient, and include, for example, conventional excipients such as antioxidants, stabilizers, solubilizers, vitamins, pigments and fragrances, and carriers.

The cosmetic composition of the present invention may also be prepared in any formulation commonly prepared in the art, and may be formulated into cosmetics, such as skin toner, astringent toner, nourishing toner, nourishing cream, massage cream, essence, eye cream, eye essence, cleansing cream, cleansing foam, cleansing water, pack, gel, powder, body lotion, body cream, body oil, and body essence.

When the formulation of the present invention is the paste, cream, or gel, as the carrier ingredient, animal oils, vegetable oils, wax, paraffin, starch, tragacanth, cellulose derivatives, polyethylene glycol, silicone, bentonite, silica, talc, zinc oxide or the like may be used.

When the formulation of the present invention is the powder or spray, as the carrier ingredient, lactose, talc, silica, aluminum hydroxide, calcium silicate or polyamide powder may be used, and particularly, in the case of the spray, a propellant such as chlorofluorohydrocarbon, propane/butane or dimethyl ether may be additionally included.

When the formulation of the present invention is the solution or emulsion, as the carrier ingredient, a solvent, a solubilizing agent or an emulsifying agent may be used. For example, the carrier ingredient includes water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or sorbitan fatty acid ester.

When the formulation of the present invention is the suspension, as the carrier ingredient, a liquid diluent such as water, ethanol or propylene glycol, a suspension such as ethoxylated isostearyl alcohol, polyoxyethylene sorbitol ester and polyoxyethylene sorbitan ester, microcrystalline cellulose, aluminum metahydroxide, bentonite, agar, tragacanth, or the like may be used.

When the formulation of the present disclosure is the surfactant-containing cleansing, as the carrier ingredient, aliphatic alcohol sulfate, aliphatic alcohol ether sulfate, sulfosuccinic acid monoester, isethionate, imidazolinium derivatives, methyltaurate, sarcosinate, fatty acid amide ether sulfate, alkylamido betaine, aliphatic alcohol, fatty acid glyceride, fatty acid diethanolamide, vegetable oil, lanolin derivatives, ethoxylated glycerol fatty acid ester, or the like may be used.

Yet another aspect of the present invention may provide a fermented Perilla frutescens extract-containing pharmaceutical composition for preventing or treating stress-related disorders, depression or anxiety.

The fermented Perilla frutescens extract is the same as that used in the antidepressant, anti-anxiety or tension-relieving composition, and thus the description is replaced with the disclosure thereof.

The stress-related disorders of the present invention may be preferably at least one selected from the group consisting of stress tension, stress anxiety, stress depression, fatigue syndrome, fear memory, mental panic attack, obsessive-compulsive disorder, post-traumatic stress disorder, schizophrenia, stress headaches, neurodegenerative diseases, eating disorders, anorexia nervosa, drug addiction, drug or alcohol withdrawal syndromes, and stress psychotic episodes, and more preferably stress tension, stress anxiety, and stress depression.

The pharmaceutical composition has an effect of preventing or treating depression or anxiety caused by various causes, but preferably, may have an effect of preventing or treating depression or anxiety caused by stress.

The pharmaceutical composition of the present invention may be various oral or parenteral formulations. When the composition is formulated, the formulations may be prepared by using one or more buffers (e.g., saline or PBS), antidiabetic agents, bacteriostatic agents, chelating agents (e.g., EDTA or glutathione), fillers, extenders, binders, adjuvants (e.g., aluminum hydroxide), suspending agents, thickening agents, wetting agents, disintegrants or surfactants, and diluents or excipients.

Solid formulations for oral administration include tablets, pills, powders, granules, capsules, etc., and these solid formulations may be prepared by mixing one or more compounds with at least one excipient, for example, starch (including corn starch, wheat starch, rice starch, potato starch, etc.), calcium carbonate, sucrose, lactose, dextrose, sorbitol, mannitol, xylitol, erythritol, maltitol, cellulose, methyl cellulose, sodium carboxymethyl cellulose and hydroxypropyl methyl cellulose or gelatin. For example, tablets or sugar-coated tablets may be obtained by mixing an active ingredient with a solid excipient, pulverizing the mixture, adding a suitable adjuvant, and then processing the mixture into a granule mixture. Further, lubricants such as magnesium stearate, talc, and the like may also be used in addition to simple excipients.

Liquid formulations for oral administration may correspond to suspensions, oral liquids, emulsions, syrups, and the like, and may include various excipients, such as a wetting agent, a sweetener, an aromatic agent, a preserving agent, or the like, in addition to water and liquid paraffin which are commonly used as simple diluents. In addition, in some cases, cross-linked polyvinylpyrrolidone, agar, alginic acid or sodium alginate may be added as a disintegrant, and an anti-coagulant, a lubricant, a wetting agent, a fragrance, an emulsifier, a preservative, and the like may be further included.

Formulations for parenteral administration include a sterile aqueous solution, a non-aqueous solution, a suspension, an emulsion, a lyophilizing agent, a suppository, or the like. As the non-aqueous solution and the suspension, propylene glycol, polyethylene glycol, vegetable oil such as olive oil, injectable ester such as ethyl oleate, and the like may be used. As a base of the suppository, witepsol, macrogol, Tween 61, cacao butter, laurinum, glycerol, gelatin, and the like may be used.

The pharmaceutical composition of the present invention may be administered orally or parenterally, and when administered parenterally, the pharmaceutical composition may be formulated in the form of external skin uses; injectable agents for intraperitoneal, rectal, intravenous, intramuscular, subcutaneous, intrauterine, epidural or intracerebrovascular injection; a transdermal agent; or a nasal inhalant according to methods known in the art.

The injectable agents need to be sterilized and protected from contamination of microorganisms such as bacteria and fungi. Examples of suitable carriers for injectable agents are not limited thereto, but may be solvents or dispersion media containing water, ethanol, polyols (e.g., glycerol, propylene glycol, and liquid polyethylene glycols), mixtures thereof and/or vegetable oils. More preferably, as the suitable carriers, a Hanks' solution, a Ringer's solution, a phosphate buffered saline (PBS) containing triethanol amine or sterile water for injection, an isotonic solution such as 10% ethanol, 40% propylene glycol and 5% dextrose, and the like may be used. In order to protect the injectable agents from microbial contamination, various antibacterial and antifungal agents such as paraben, chlorobutanol, phenol, sorbic acid, thimerosal, and the like may be further included. In addition, most of the injectable agents may further include an isotonic agent, such as sugar or sodium chloride.

The transdermal agents are included in the form of ointments, creams, lotions, gels, external liquids, pastas, liniments, and aerosols. Hereinabove, the transdermal administration means that an effective amount of active ingredient contained in the pharmaceutical composition is delivered into the skin by topically applying the pharmaceutical composition to the skin.

In the case of the inhalants, the extract used according to the present invention may be conveniently delivered in the form of an aerosol spray from a pressurized pack or nebulizer by using a suitable propellant, such as dichlorofluoromethane, trichlorofluoromethane, dichlorotetrafluoroethane, and carbon dioxide, or other suitable gases. In the case of the pressurized aerosol, a dosage unit may be determined by providing a valve that delivers a measured amount. For example, gelatin capsules and cartridges used in an inhaler or insufflator may be formulated to contain a powder mixture of a compound and a suitable powder base such as lactose or starch. Formulations for parenteral administration may be formulations generally known in all pharmaceutical chemistries.

The pharmaceutical composition of the present invention is administered in a pharmaceutically effective amount, and the pharmaceutically effective amount refers to an amount enough to treat a disease at a reasonable benefit/risk ratio applicable to medical treatment. An effective dose level may be determined according to factors including the type and severity of a patient's disease, the activity of a drug, the sensitivity to a drug, a time of administration, a route of administration, an excretion rate, duration of treatment, and simultaneously used drugs, and other factors well-known in the medical field. The composition of the present invention may be administered as an individual therapeutic agent or in combination with other therapeutic agents, and may be administered sequentially or simultaneously with conventional therapeutic agents, and administered singly or multiply. That is, the total effective amount of the composition of the present invention may be administered to a patient in a single dose, or may be administered according to a fractionated treatment protocol, which is administered in a multiple dose for a long period of time. It is important to administer an amount capable of obtaining a maximum effect with a minimal amount without side effects by considering all the factors and this may be easily determined by those skilled in the art.

The dose of the pharmaceutical composition of the present invention varies in the range thereof according to the weight, age, sex, health condition, diet, administration time, administration method, and excretion rate of a patient, the severity of disease, etc. In the parenteral administration, the daily dose may be administered in an amount of preferably 0.01 to 200 mg, and more preferably 0.1 to 120 mg per 1 kg of body weight per day based on fermented Perilla frutescens extract. In addition, in the oral administration, the daily dose may be administered one or several times in an amount of preferably 0.01 to 1,000 mg, and more preferably 0.1 to 500 mg per 1 kg of body weight per day based on the fermented Perilla frutescens extract of the present invention. However, since the dose may increase or decrease depending on the route of administration, the severity of obesity, sex, body weight, age, etc., the dose does not limit the scope of the present invention in any way.

The composition of the present invention may be used alone or in combination with surgery, radiation therapy, hormone therapy, chemotherapy, and methods of using biological response modifiers.

The pharmaceutical composition of the present invention may also be provided in the form of an external preparation containing a fermented Perilla frutescens extract as an active ingredient. When the pharmaceutical composition of the present invention for stress-related disorders, depression or anxiety is used as an external skin preparation, the pharmaceutical composition may further contain adjuvants commonly used in the field of dermatology, such as any other ingredients commonly used in external skin preparations, such as fatty substances, organic solvents, solubilizers, thickening and gelling agents, emollients, antidiabetic agents, suspending agents, stabilizers, foaming agents, fragrances, surfactants, water, ionic emulsifiers, nonionic emulsifiers, fillers, metal ion sequestrants, chelating agents, preservatives, vitamins, blockers, wetting agents, essential oils, dyes, pigments, hydrophilic activators, lipophilic activators or lipid vesicles. In addition, the ingredients may be introduced in an amount generally used in the field of dermatology.

When the pharmaceutical composition of the present invention for stress-related diseases, depression or anxiety is provided as an external skin preparation, the external skin preparation may be a formulation such as an ointment, a patch, gel, cream, or spray, but is not limited thereto.

Still another aspect of the present invention may provide a fermented Perilla frutescens extract-containing dietary supplement composition for preventing or ameliorating stress-related disorders, depression or anxiety.

The fermented Perilla frutescens extract is the same as that used in the antidepressant, anti-anxiety or tension-relieving composition, and thus the description is replaced with the disclosure thereof.

Since the stress-related disorders are the same as the stress-related disorders targeted by the pharmaceutical composition, the description is replaced with the disclosure thereof.

The dietary supplement composition has an effect of preventing or ameliorating depression or anxiety caused by various causes, but preferably, may have an effect of preventing or ameliorating depression or anxiety caused by stress.

The kind of dietary supplement is not particularly limited. Examples of the dietary supplement include drinks, meat, sausages, bread, biscuits, rice cakes, chocolate, candies, snacks, confectionery, pizza, ramen, other noodles, gums, dairy products including ice cream, various soups, beverages, alcohol beverages and vitamin complex, dairy products and processed dairy products, and the like, and include all dietary supplements in an accepted meaning.

The fermented Perilla frutescens extract of the present invention may be added to the food as it is or used with other foods or food ingredients, and may be appropriately used according to a general method. A mixing amount of the active ingredients may be suitably determined according to the purpose of use thereof (for prevention or amelioration). In general, the amount of the compound in the health food may be added in an amount of 0.1 to 90 parts by weight of the total food weight. However, in the case of long-term intake for the purpose of health and hygiene or for the purpose of health control, the amount may be equal to or greater than the range, and there is no problem in terms of safety, so that the active ingredients may be used even in an amount above the range.

A health functional beverage composition of the present invention is not particularly limited to other ingredients in addition to containing the compound of the present invention as an essential ingredient in an indicated ratio, and may contain various flavoring agents or natural carbohydrates as an additional ingredient, like conventional beverages. Examples of the above-described natural carbohydrates include conventional sugars, including monosaccharides, such as glucose, fructose, etc.; disaccharides, such as maltose, sucrose, etc.; and polysaccharides, such as dextrin, cyclodextrin, etc., and sugar alcohols such as xylitol, sorbitol, erythritol, etc. As flavoring agents other than those described above, natural flavoring agents (tauumatin, stevia extract (e.g., rebaudioside A, glycyrhizin, etc.)), and synthetic flavoring agents (saccharin, aspartame, etc.) may be advantageously used. A ratio of the natural carbohydrates may be generally about 1 to 20 g, preferably about 5 to 12 g per 100 of the composition of the present invention.

In addition, the fermented Perilla frutescens extract of the present invention may contain various nutrients, vitamins, minerals (electrolytes), flavoring agents such as synthetic and natural flavoring agents, coloring agents, and enhancers (cheese, chocolate, etc.), pectic acid and salts thereof, alginic acid and salts thereof, organic acid, a protective colloidal thickener, a pH adjusting agent, a stabilizer, a preservative, glycerin, alcohol, a carbonic acid agent used in a carbonated drink, etc. In addition, the fermented Perilla frutescens extract of the present invention may contain fruit pulp for the production of natural fruit juice, fruit juice beverages, and vegetable beverages. These ingredients may be used independently or in combination. The ratio of these additives is not that important, but is generally selected in the range of 0.1 to about 20 parts by weight per 100 parts by weight of the fermented Perilla frutescens extract of the present invention.

Yet another aspect of the present invention provides a fermented Perilla frutescens extract-containing fiber or fragrance composition for relieving depression, anxiety, tension, or stress.

The fermented Perilla frutescens extract is the same as that used in the antidepressant, anti-anxiety or tension-relieving composition, and thus the description is replaced with the disclosure thereof.

The depression, anxiety, or tension is the same as that targeted by the antidepressant, anti-anxiety or tension-relieving composition, and thus the description is replaced with the disclosure thereof.

The fiber of the present invention refers to all fibers that exhibit a depression, anxiety, tension or stress-relieving effect due to the fermented Perilla frutescens extract, such as being prepared using the fermented Perilla frutescens extract as a raw material, being dyed using the fermented Perilla frutescens extract as a dye, or attaching the fermented Perilla frutescens extract on the surface of the fiber by spraying, etc., and preferably a fiber dyed using the fermented Perilla frutescens extract as a dye.

The fiber may be used in all products that may affect the living environment, and preferably, is included in at least one product selected from the group consisting of bedding, clothing, curtains, carpets, indoor shoes, towels, wallpaper, indoor coverings made of textile materials, dolls, and eye masks, but is not limited thereto.

The bedding refers to bedding (comforters, mattresses), pillows or cushions necessary for sleeping, the clothing refers to pajamas, underwear, or socks that may be worn while sleeping, and the indoor coverings made of textile materials refer to textile-made covers used to cover and store furniture such as chairs or desks, decorations, and equipment due to contamination or other reasons, but are not limited thereto.

The fragrance of the present invention refers to all materials used to produce fragrance, such as aroma and the like.

The fragrance composition may be included without limitation in any product that may be used for giving off the scent while exhibiting an effect of relieving depression, anxiety, tension or stress due to the fermented Perilla frutescens extract, but preferably included in at least one product selected from the group consisting of perfumes, scented candles, oils, air fresheners, detergents, and external skin preparations.

The external skin preparations include all products necessary for bathing, such as soap, cleansers, or bath agents, but are not limited thereto.

The amount of the fermented Perilla frutescens extract included in the products may be appropriately adjusted and included to achieve a desired effect according to conventional techniques in the art.

Still another aspect of the present invention provides a method for treating stress-related disorders, depression or anxiety, including administering a fermented Perilla frutescens extract to a patient suffering from stress-related disorders, depression, or anxiety.

The fermented Perilla frutescens extract is the same as that used in the antidepressant, anti-anxiety or tension-relieving composition, and thus the description is replaced with the disclosure thereof.

Since the stress-related disorders are the same as the stress-related disorders targeted by the pharmaceutical composition, the description is replaced with the disclosure thereof.

### Advantageous Effects

Therefore, according to the present invention, a fermented Perilla frutescens (Perilla frutescens Britton var. acuta Kudo (Chajgi), or Perilla frutescens Britton var. crispa Decaisn (Jasoyeop)) extract significantly relieves stress compared to an unfermented Perilla frutescens extract, and thus can be used as an antidepressant, anti-anxiety, anti-stress, tension-relieving or stress-relieving composition, and can also be used as a composition for preventing, ameliorating, or treating stress-related disorders, depression, and anxiety. Furthermore, the fermented Perilla frutescens extract can also be effectively used as a method for treating stress-related disorders, depression or anxiety by including administering the fermented Perilla frutescens extract to a patient suffering from stress-related disorders, depression, or anxiety.

### DESCRIPTION OF DRAWINGS

FIG. 1 shows results of measuring the immobility time of mice through a tail suspension test (TST) after administering a Perilla frutescens extract (Cha W) or fermented Perilla frutescens extracts (Cha B and Cha L) to mice induced with stress by restraint (restrict stress). It was confirmed that the immobility time was significantly reduced when Cha B or Cha L was administered compared to Cha W.

FIG. 2 shows results of measuring the immobility time of mice through a forced swimming test (FST) after administering a Perilla frutescens extract (Cha W) or fermented Perilla frutescens extracts (Cha B and Cha L) to mice induced with stress by restraint (restrict stress). It was confirmed that the immobility time was significantly reduced when Cha B or Cha L was administered compared to Cha W.

FIG. 3 shows results of measuring the immobility time of mice through a tail suspension test (TST) after administering a Perilla frutescens extract (Cha W) or fermented Perilla frutescens extracts (Cha B and Cha L) to mice induced with stress by sleep deprivation (sleep deprivation). It was confirmed that the immobility time was significantly reduced when Cha B or Cha L was administered compared to Cha W.

FIG. 4 shows results of measuring the immobility time of mice through a forced swimming test (FST) after administering a Perilla frutescens extract (Cha W) or fermented Perilla frutescens extracts (Cha B and Cha L) to mice induced with stress by sleep deprivation (sleep deprivation). It was confirmed that the immobility time was significantly reduced when Cha B or Cha L was administered compared to Cha W.

### MODES OF THE INVENTION

### Example 1

Preparation of Perilla frutescens Extract and Fermented Product Thereof

A hot aqueous extract of Perilla frutescens was prepared by extracting Perilla frutescens with hot water at 80° C. and sterilizing the extract at 121° C. for 20 minutes (Cha W). The sterilized hot aqueous extract of Perilla frutescens was fermented with Bacillus or Lactobacillus bacteria to prepare a fermented Perilla frutescens extract.

Specifically, the Perilla frutescens extract was inoculated with Bacillus subtilis as the Bacillus bacteria or Lactobacillus rhamnosus as the Lactobacillus bacteria, and then shaking-cultured at 140 rpm for 5 days in an environment of 25° C. and 80% humidity to finally prepare fermented Perilla frutescens extracts of a fermented product of Perilla frutescens Bacillus subtilis (Cha B) and a fermented product of Perilla frutescens Lactobacillus rhamnosus (Cha L).

### Example 2

### Induction of Depression, Anxiety, Tension or Stress in Mice

### <2-1> Induction of Depression, Anxiety, Tension or Stress by Restraint

ICR mice (Orient Bio Co., Ltd.) were orally administered once with 0.9% saline solution (CTL, Veh) and 100 mg/kg of the Perilla frutescens extract (Cha W) or its fermented extract (Cha B, Cha L) prepared in Example 1, and after 30 minutes, added in a circular restraint frame every 3 hours for 5 days to induce restraint stress. After the stress induction was completed, the mice were stabilized for 24 hours, and then subjected to a tail suspension test (TST) or a forced swimming test (FST) to measure the immobility time of the mice, respectively.

### <2-2> Induction of Depression, Anxiety, Tension or Stress by Sleep Deprivation

After water was filled in a tank where a platform was erected, ICR mice (Orient Bio Co., Ltd.) were placed on the platform and then sleep-deprived for 3 days. At this time, 100 mg/kg of 0.9% saline solution (CTL, Veh), or the Perilla frutescens extract (Cha W) or its fermented extract (Cha B, Cha L) prepared in Example 1 was orally administered once at a set time (10:00 AM) for 3 days. After the stress induction was completed, the mice were stabilized for 24 hours, and then subjected to a tail suspension test (TST) or a forced swimming test (FST) to measure the immobility time of the mice, respectively.

### Example 3

Confirmation of Effects of Fermented Perilla frutescens Extract

### <3-1> Tail Suspension Test

After stress was induced in Example 2, a tape was attached to the tail end of the stabilized mouse and the mouse was hung upside down on the wall. Movement was observed for a total of 6 minutes, and the levels of stress, etc. were measured by measuring the time in a motionless and immobile condition.

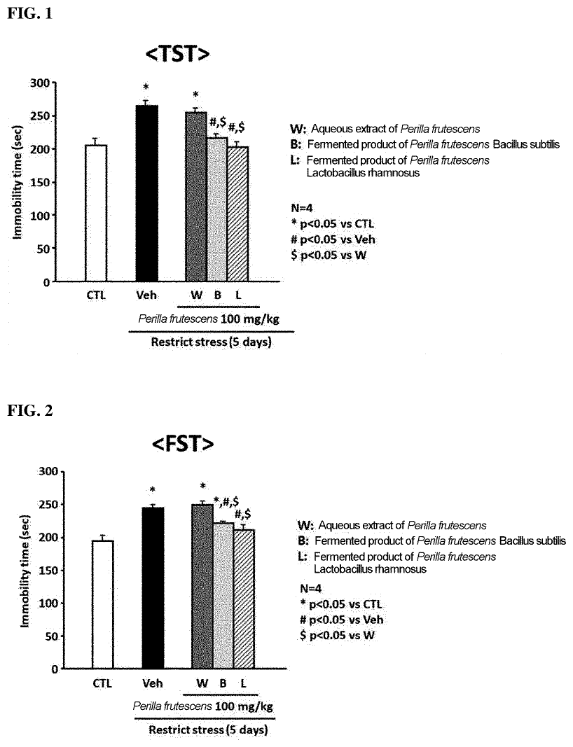

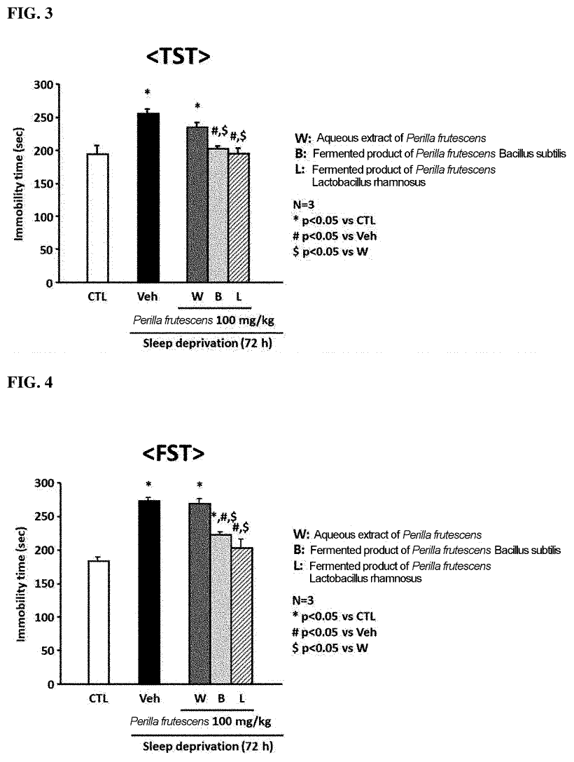

As a result, the immobility time of mice induced with stress and the like by restraint (FIG. 1) or sleep deprivation (FIG. 3) was significantly increased (Veh). In addition, when the unfermented Perilla frutescens extract (Cha W) was administered to the mice, there was no change in the immobility time, but when the fermented Perilla frutescens extract (Cha B or Cha L) was administered, it was confirmed that the immobility time was significantly reduced.

### <3-2> Forced Swimming Test

After filling a water in a tank of about 30 cm, mice stabilized after inducing the stress in Example 2 above were placed therein. Movement was observed for a total of 7 minutes, and the levels of stress, etc. were measured by measuring the time in a motionless and immobile condition.

As a result, the immobility time of mice induced with stress and the like by restraint (FIG. 2) or sleep deprivation (FIG. 4) was significantly increased (Veh). In addition, when the unfermented Perilla frutescens extract (Cha W) was administered to the mice, there was no change in the immobility time, but when the fermented Perilla frutescens extract (Cha B or Cha L) was administered, it was confirmed that the immobility time was significantly reduced.

### [Preparation Example]

### 1. Preparation of Health Functional Beverage

| | | |
|---|---|---|
| Vitamin | 0.5 | wt % |
| Dietary fiber | 4.0 | wt % |
| High fructose corn syrup | 93.0 | wt % |
| Emulsifier | 1.0 | wt % |
| Fragrance | 0.5 | wt % |
| Fermented Perilla frutescens extract | 1.0 | wt % |

The composition was mixed with purified water to have the total volume of 50 ml. The mixture passed through a 2 to 3-μm filter to remove floating matters, and then the final mixture was sterilized at 90 to 93° C. for 15 to 20 seconds, filled into a 50 ml bottle, and sterilized at 80 to 85° C. for 15 to 20 minutes to complete a health functional beverage product.

### 2. Preparation of Skin Toner

| | | |
|---|---|---|
| Hydroxyethylene cellulose (2% aqueous solution) | 12.0 | wt % |
| Xanthan gum (2% aqueous solution) | 2.0 | wt % |
| 1,3-butylene glycol | 6.0 | wt % |
| Glycerin | 4.0 | wt % |
| Sodium hyaluronate (1% aqueous solution) | 5.0 | wt % |
| Ion exchange water | 73.0 | wt % |
| Fermented Perilla frutescens extract | 3.0 | wt % |

The toner was prepared using a conventional toner preparation method.

### 3. Preparation of Air Freshener

| | |
|---|---|
| 95% ethanol | 65.0 to 75.0 wt % |
| Fermented Perilla frutescens extract | 25.0 to 35.0 wt % |

The ethanol and the fermented Perilla frutescens extract were mixed and then stirred at room temperature for 12 to 20 minutes to prepare an air freshener.

### 4. Preparation of Bath Agent

| | | |
|---|---|---|
| Sodium bicarbonate | 70.0 | wt % |
| Anhydrous sodium sulfate | 29.0 | wt % |
| Fermented Perilla frutescens extract | 1.0 | wt % |

The sodium bicarbonate and the anhydrous sodium sulfate were stirred with a V-type mixer until uniform, and then added with the fermented Perilla frutescens extract and stirred sufficiently again until uniform to prepare a bath agent.

### INDUSTRIAL APPLICABILITY

A fermented Perilla frutescens extract according to the present invention significantly relieves stress compared to an unfermented Perilla frutescens extract as a natural extract, and thus can be effectively used as an antidepressant, anti-anxiety, anti-stress, tension-relieving or stress-relieving composition, and can also be used as a composition for preventing, ameliorating, or treating stress-related disorders, depression, and anxiety. Furthermore, the fermented Perilla frutescens extract can also be used as a method for treating stress-related disorders, depression or anxiety by including administering the fermented Perilla frutescens extract to a patient suffering from stress-related disorders, depression, or anxiety, and thus has high industrial applicability.

1. An antidepressant, anti-anxiety, anti-stress, or tension-relieving composition comprising a fermented Perilla frutescens extract.

2. The antidepressant, anti-anxiety, anti-stress, or tension-relieving composition of claim 1, wherein the fermented extract is extracted from at least one part selected from the group consisting of leaves, stems, flowers, fruits, and whole plant of Perilla frutescens.

3. The antidepressant, anti-anxiety, anti-stress, or tension-relieving composition of claim 1, wherein the fermented extract is extracted using water, an organic solvent, or a mixture thereof as a solvent and then fermented with a fermentation strain.

4. The antidepressant, anti-anxiety, anti-stress, or tension-relieving composition of claim 3, wherein the fermentation strain is Bacillus or Lactobacillus bacteria.

5. The antidepressant, anti-anxiety, anti-stress, or tension-relieving composition of claim 4, wherein the Bacillus bacteria are at least one strain of the genus Bacillus selected from the group consisting of Bacillus subtilis, Bacillus thuringiensis, Bacillus licheniformis, Bacillus amyloliquefaciens, Bacillus stearothermophilus, Bacillus coagulans, Bacillus longum, Bacillus pumilus, Bacillus brevis, Bacillus circulans, and Bacillus polymyxa; and
the Lactobacillus bacteria are at least one strain of the genus Lactobacillus selected from the group consisting of Lactobacillus acidophilus, Lactobacillus casei, Lactobacillus gasseri, Lactobacillus bulgaricus, Lactobacillus helveticus, Lactobacillus fermentum, Lactobacillus paracasei, Lactobacillus plantarum, Lactobacillus reuteri, Lactobacillus rhamnosus, and Lactobacillus salivarius.

6. The antidepressant, anti-anxiety, anti-stress, or tension-relieving composition of claim 3, wherein the fermentation is performed at 5° C. to 80° C. for 30 minutes to 10 days.

7. The antidepressant, anti-anxiety, anti-stress, or tension-relieving composition of claim 1, wherein the depression, anxiety or tension is caused by stress.

8. (canceled)

9. The composition of claim 1, wherein the composition is at least one composition selected from the group consisting of a food composition, a cosmetic composition, a dye composition, a fragrant composition, a pharmaceutical composition, and a quasi-drug composition.

10-12. (canceled)

13. A dietary supplement composition comprising a fermented Perilla frutescens extract for preventing or ameliorating stress-related disorders, wherein the stress-related disorders are at least one selected from the group consisting of stress tension, stress anxiety, stress depression, fatigue syndrome, fear memory, mental panic attack, obsessive-compulsive disorder, post-traumatic stress disorder, schizophrenia, stress headaches, neurodegenerative diseases, eating disorders, anorexia nervosa, drug addiction, drug or alcohol withdrawal syndromes, and stress psychotic episodes.

14. The dietary supplement composition of claim 13, wherein the stress-related disorders are at least one selected from the group consisting of stress tension, stress anxiety, stress depression, fatigue syndrome, fear memory, mental panic attack, obsessive-compulsive disorder, post-traumatic stress disorder, schizophrenia, stress headaches, neurodegenerative diseases, eating disorders, anorexia nervosa, drug addiction, drug or alcohol withdrawal syndromes, and stress psychotic episodes.

15-20. (canceled)

21. A method for treating depression, anxiety or stress-related disorders, comprising administering a fermented Perilla frutescens extract to a patient suffering from depression, anxiety or stress-related disorders.

22. (canceled)
